(19) United States
(12) Patent Application Publication
(10) Pub. No.: US 2016/0242731 A1
(43) Pub. Date: Aug. 25, 2016

(54) Smart blood pressure measuring system (SBPMS)

(71) Applicant: Albrik Levick Gharibian, Glendale, CA (US)

(72) Inventor: Albrik Levick Gharibian, Glendale, CA (US)

(21) Appl. No.: 14/757,077

(22) Filed: Nov. 16, 2015

Related U.S. Application Data
(60) Provisional application No. 62/124,355, filed on Dec. 17, 2014.

(51) Int. Cl. A61B 7/04; A61B 5/00; A61B 5/021

(52) U.S. Cl. CPC A61B 7/045; A61B 5/021; A61B 5/0082; A61B 5/6803; A61B 5/6898

(57) ABSTRACT

A new method for measuring blood pressure using a small sound measuring device that communicates wirelessly with any mobile device (i.e. smart phone). The device measures the sound of the blood flowing through an artery from any number of possible locations on a person's body. This sound measurement is then communicated wirelessly to the person's mobile device and is picked up by a downloaded mobile software application that is open to listen for the device's signal. The application then takes that sound measurement and compares it against a stored database of pre-measured sound to blood pressure measurements. The application then outputs to the user their current blood pressure measurement.

This invention generally relates to a Non-Invasive blood pressure measuring system that captures the sound generated from the blood flow through artery by using a Sound Measuring Device (SMD).

This measured sound in “Deci-Bells” (dB) is compared to typical measured blood pressure. This means, the measured sound generated from the flow of blood through an artery will be equal to the typical cuff measured blood pressure. For example, typical cuff blood measured (Systolic/Diastolic) was 120/80 and the sound measured for the same 120/80 blood pressure was 1 bB. This would mean that 120/80 would represented as 1 dB sound level. Therefore, 1 dB measured sound will be displayed as 120/80 Systolic/Diastolic blood pressure.

A normal range of typically recorded Systolic/Diastolic blood pressure in mm Hg, (Millimeters of mercury) will be recorded. At the same time the sound generated from flow of blood through an artery is measured for each typical Systolic and Diastolic blood pressure measurements. This range will include all the measurements expected from a typical cuff measured Sphygmomanometer blood pressure.

The data obtained from this range of measurements between the typical Systolic and Diastolic vs. the equated sound will be calibrated accordingly and tabulated in a database. This database will be formulated in an APP. (Application), where the free download will be available for any Mobile device with Android or Apple mobile Operating System, (MOS).

The Smart Blood Pressure measuring System will measure the sound captured from blood flowing through an artery, for example 1 dB sound level. The mobile device will have the downloaded Application with the range of measured database. This 1 dB measured sound level, for example, will be equated to the 120/80, where this number will be displayed on the Mobile device. This number will be generated from the downloaded APP with a built in formatted query.

The measured sound level obtained from Sound Measuring Device (SMD) will be transmitted to the downloaded Application database of the Mobile device wirelessly. The measured sound value that is equal to the pre-measured blood pressure number will be displayed on the Mobile device.

The following is a sample of a Tabulated database in the form of a table. This table with filtering and sorting formulation will be part of the downloaded application in any mobile device.

The following table contains arbitrary examples of measured values, where by input sound to the mobile device vs. the equivalent cuff inflated blood measured from the application will be displayed as the output on a mobile device.

| Measured sound by Sound | Output blood pressure displayed on a |
|---|---|
| Measuring Device becomes the | mobile device by comparing to its |
| input to the mobile device | equivalent measured input sound |
| 0.09 dB | 115/85 mm/Hg |
| 0.1 dB | 120/80 mm/Hg |
| 0.2 dB | 125/75 mm/Hg |
| 0.3 dB | 130/70 mm/Hg |

Systolic is the upper number of the pressure in the arteries when the heart beats or the heart muscle contracts.

Diastolic is the lower number, where the heart muscle is resting between beats.

The sound level measured from artery will be higher indicating the Systolic number and lower, corresponding to the Diastolic number.

My Smart Blood Pressure Measuring System (SBPMS) eliminates the current cuff blood measuring system. This can be achieved by having a separate or built in Sound Measuring Device (SMD) to a Mobile device, as noted below.

The Sound Measuring Device (SMD) can be separate unit. This device can be as small as a hearing aid and can be put inside an ear to measure the blood flow through the dr Wry that exist in ears, OR, the Sound Measuring Device (SMD) can be built in the future Mobile devices. The Mobile device then can be used to detect the sound level generated from the artery of the wrist. This can be achieved by having the Mobile device as close as to the skin of the wrist, in order to measure the sound level of blood flow through an artery.

Currently a typical cuff-style “Wrist Digital Blood Pressure Monitor” is attached for measuring blood pressure.

### BACKGROUND

### Prior Art

Currently, Cuff-Style (Sphygmomanometer) or a “Wrist Digital Blood Pressure Monitor” measures blood pressure. This device uses the inflated cuff that measures the Systolic and Diastolic blood pressure. However, my invention does not use the inflated cuff-style to measure blood pressure.

My Smart Blood Pressure Measuring System (SBPMS) uses the sound generated from the flow of blood through an artery in three different ways:
1. A separate ear bud style Sound Measuring Device (SMD)
2. A built in SMD in a Mobile device
3. A miniaturized completely separate Sound Measuring Device that can be attached to the input jack of any Mobile device.

### BRIEF DESCRIPTION OF DRAWINGS

FIG. 1:

#4. Sound Measuring Device (SMD) in the shape of an ear bud, inside ear, measures the sound generated from the blood through artery, inside ears.

#5. Represents a Mobile Device with loaded application, that wirelessly receives measured sound from SMD. The application sorts the equivalent blood pressure by indicating 120/80 mm/Hg

#6 Wireless connection between the Mobile device and the SMD

The Blood pressure 120/80 is indicated after the ear bud transmits the measured sound wirelessly to the Application. The application associates the measured received sound to the corresponding blood pressure and as shown it indicated the correct blood pressure, for example as shown 120/80.

Ear bud can be placed inside an ear to measure the sound level in an Artery. This will replace the common cuff inflated blood pressure measuring method.

FIG. 2:

#4. Integrated SMD inside the Mobile Device

#5. Represents a Mobile Device with loaded Application and integrated SMD that touches the hand artery. Measures the sound of blood flowing through the artery, indicating blood pressure, (BP) 120/80 mm/Hg

#7. Human hand with the Mobile Device's SMD touching the artery and measuring the Sound level through artery

FIG. 2. Indicates a mobile device with built in Sound Measuring Device (SMD) and Loaded Application. This Mobile device will touch artery in the wrist and measure the sound. This measured sound will be linked to the associated blood pressure and will indicate on the Mobile device, for example 120/80.

FIG. 3:

#4. The external Sound Measuring Device (SMD) connected to the Mobile device via the input Jack of the Mobile device. This will touch the arm artery, to measure the blood sound through the artery.

#5. A Mobile device connected to a Sound measuring device (SMD) via the input Jack of the Mobile device with the loaded Application. Measuring the sound from wrist artery. Indicating blood pressure of 120/80

#7. Human hand with the Mobile Device's SMD touching the artery and measuring the Sound level through artery

FIG. 3. Indicates the separate Sound Measuring Device (SMD) fitted into the input Jack of the Mobile device with the App. The Mobile device shows the associated blood pressure of 120/80

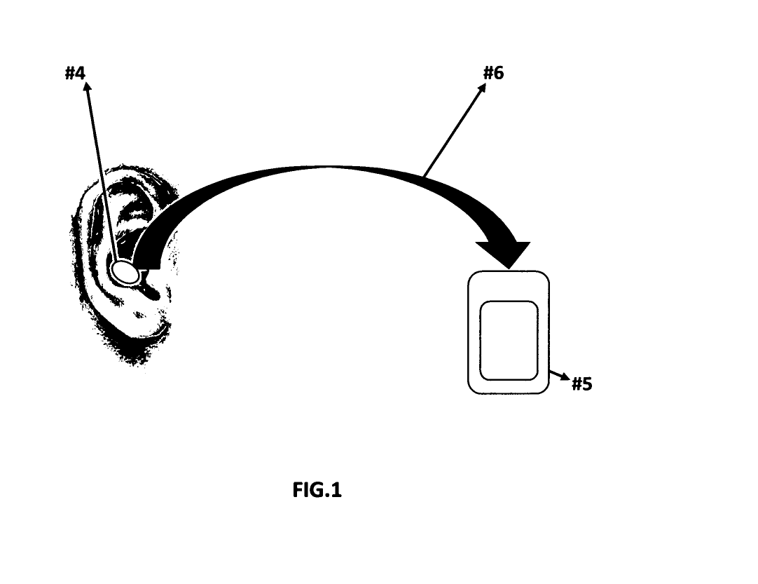

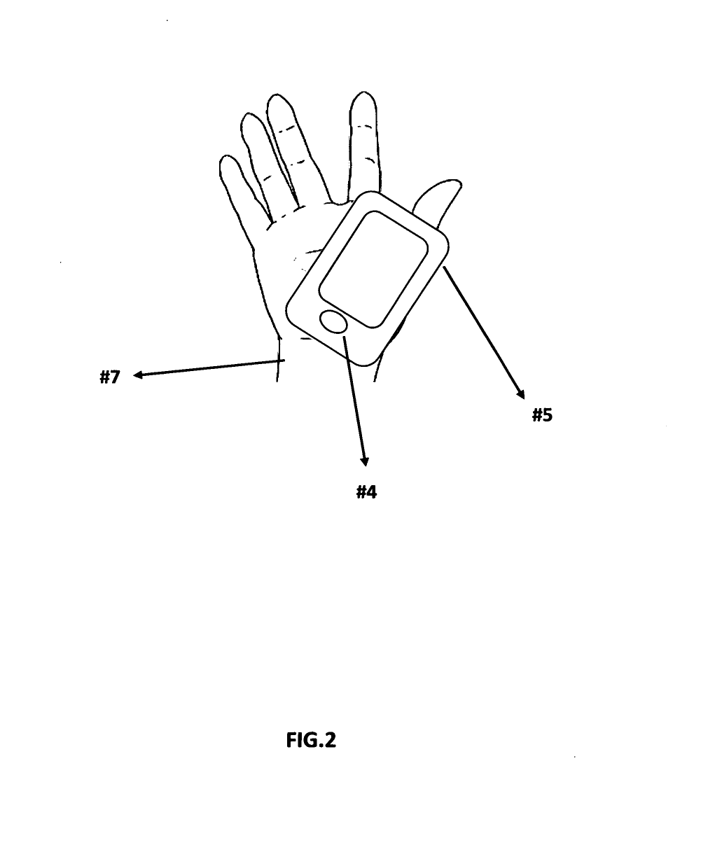

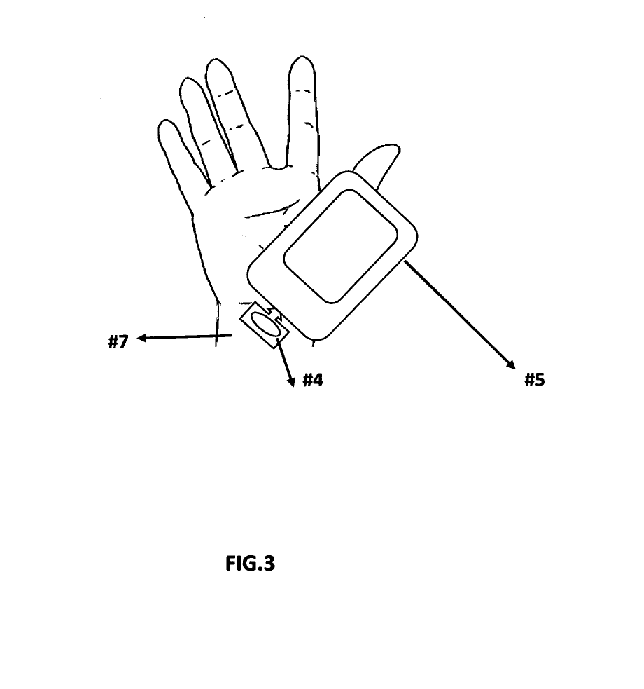

Summary of the three types of Sound Measuring Devices (SMD) that can be used with my idea, not excluding other possible and compatible types:
1. This device, namely, “Sound Level Meter” (SMD) can be as small as the size of a hearing aid and it can be fitted in an ear, in the form of an ear bud. This device will detect the sound from the blood flowing through artery in an ear. This measurement will be transmitted wirelessly to a Mobile device. As the Mobile device's “Blood Pressure Measuring” Application (APP) is selected, the sound measured by the “Sound Level Meter” will be the input to the “Blood Measuring Application” on the Mobile device. This measured sound will be the input to the formulated downloaded Application that contains the database. By comparing the input received from the sound measured with the pre-tabulated blood pressure from the database, the result will be displayed on the Mobile device. For example, 134/98 Or 120/80. Please refer to the FIG. 1.
2. The Sound Measuring Device (SMD) could be built in to a Mobile device. In this case, in order to measure the blood pressure, the Mobile device can be brought close to touching the artery of the wrist, where blood flows through the artery. The sound generated can be measured using the built in Sound Measuring Device (SMD) located inside the Mobile device. The measured sound will be the input to the pre-loaded Application in the Mobile device. This measured sound will be compared with the existing database in the downloaded Application. The associated value or the number will be displayed on the Mobile device, indicating the blood pressure. For example, 134/98 or 120/80, as shown in FIG. 2.
3. The Sound Measuring device (SMD) can be manufactured such that it can be attached to the Mobile's input jack. This device like step 2 above will measure the sound from the wrist artery by touching the artery. This measured sound value again will be compared, converted into Systolic/Diastolic blood pressure in mm Hg and displayed on the Mobile device. For example, 134/98, as shown in FIG. 3.

The main distinguishable difference between the existing “Digital Blood Pressure Monitor” and my (SBPMS) is that my device uses the sound and compares to the equivalent pre-measured blood pressure that is tabulated in the downloaded Application database and displays the results on the Mobile device.

On the other hand, the existing “Digital Blood Pressure Monitor” relies on the cuff inflated measured blood pressure.

The main disadvantages of my (SBPMS) invention is that the sound measured will have some noise included in the frequency of the measured sound from the flow of the blood in an artery. This noise from the measured sound can be filtered using built in electronic sound filtering system. Therefore, the measured sound can be almost without any ambient noise.

The second disadvantage my Invention is that measuring the sound from the flow of the blood in artery will be very hard to detect. This is because the audio-range oscillations occur between 20 and 20,000 Hz. The 20 Hz corresponds to a very low pitch while 20,000 Hz corresponds to a very high pitch.

However, currently, there are different devices that will measure the sound level of various arterial pulse variations. For example, “Sound Level Meter—Type 2250” can measure sound variations starting from 4.2 Hz to 22.4 KHz broadband linear frequency range. DB (decibel) is the proportional unit of sound. The lowest sound audible with a normal (average) hearing is 0 dB.

This Sound Measuring Device, measures the sound pulsation amplitude by calibrating in terms of dBs the output from Sound Measuring Device will become the input to the downloaded Application database. The nearest and closest dB value to the Systolic/Diastolic blood pressure in mm Hg located in the database will be displayed as the blood pressure on the Mobile device.

### CONCLUSION, RAMIFICATIONS AND SCOPE

I have provided three different possibilities that my idea can implement, as shown in FIGS. 1, 2 and 3.

While the above description contain many detailed items, these should not be understood as limitations on the scope of any expression, as illustrated by examples of the presently preferred new ideas.

Many other outcomes and variations are possible within the ideas of the various expressions. For example, instead of measuring the sound level in artery, a non-invasive, and non-contact miniaturized system to measure pulse waveform of artery via applying laser triangulation method to detect skin surface vibration. This method includes a miniaturized arterial pulsation measurement system, which consists of a laser diode and a complementary metal-oxide semiconductor image sensor. For example, this system can be used instead of the Sound Measuring Device (SMD) in my examples above. Which-ever system is cost effective and technologically suitable, it can be used in conjunction with my idea.

Thus the scope of the invention should be determined by the appended claims and their legal equivalents, and not by the examples given.

1. A pre formulated Application that contains a database with all the normal pre-measured Cuff Inflated blood pressure vs the equivalent Sound Measured number using any “Sound Measuring Device” that measured the sound captured from blood flowing in the ear or wrist artery. These measurements are tabulated in the database. The sound measured will be the input to the mobile device and the Application will compare the equivalent number of the measured sound vs. the pre-measured Cuff Inflated blood pressure. The free downloaded App. compares and indicates the exact blood pressure.

2. The non-invasive “Sound Measuring Device” (SMD) measures the sound moving through an artery will be connected to the Mobile Device via the Mobile Device's input Jack. Where the (SMD) will be brought close to the wrist artery and the measured sound will be the input to the Mobile App.

3. The (SMD) can be made to a hearing aid size so that it will be placed inside an ear. The measured sound from the ear artery will be the input to the Mobile device's App.

4. If the (SMD) is integrated in to a Mobile Device then the Mobile Device will be brought close to a wrist artery and the sound measured will be the input to the downloaded APP.

5. The above claims 2, 3, and 4 will eliminate the current Cuff inflated blood measuring devices.

6. The measured sound will wirelessly be the input to the Mobile Device's downloaded Application. This input will be compared to its equivalent pre-measured Systolic and Diastolic blood pressure, which will be indicated on the Mobile Device, for example 120/80 mmHg.

7. This invention is based on the measured sound through an artery using any method of measuring the sound, either an Ultrasound or a sensitive microphone on a Mobile Device or otherwise.

8. The (SMD) could also be in the form of Laser Triangulation and a CMOS image sensor, where this non-invasive system can measure pulse waveforms of artery via applied laser triangulation method to detect skin surface vibration. This change in the skin expansion and contraction will simulate the pressure exerted by the heart in the artery. This measured change in the arterial pulsation will be equal to the Systolic and Diastolic blood pressure measurements.
